(19) United States
(12) Patent Application Publication
(10) Pub. No.: US 2023/0282372 A1
(43) Pub. Date: Sep. 7, 2023

(54) CLOUD-BASED SYSTEM FOR REAL-TIME MONITORING OF SUBJECT AND IN-VEHICLE CLOUD-BASED SYSTEM

(71) Applicant: Advatech Healthcare Europe Ltd, London (GB)

(72) Inventor: Samit Kumar Biswas, London (GB)

(21) Appl. No.: 18/175,806

(22) Filed: Feb. 28, 2023

(30) Foreign Application Priority Data
Mar. 3, 2022 (GB) GB2202964.9

(51) Int. Cl. G16H 80/00; G06V 20/59; A61B 5/00

(52) U.S. Cl. CPC G16H 80/00; A61B 5/6801; A61B 5/742; A61B 5/747; G06V 20/597

(57) ABSTRACT

Disclosed is a cloud-based system for real-time monitoring of a subject. System comprises a real-time location tracker to track location of subject; image capturing arrangement to capture an image data of subject and provide image data to a cloud server; a microphone to provide a two-way audio communication between subject and a caregiver; a wearable device to provide a real-time medical data of subject to caregiver; and a processing arrangement, operatively coupled to wearable device, to predict health condition, monitor a progression of health condition, generate an alert based on progression of health condition, and advice, based on the generated alert, an action to be performed. Moreover, real-time location tracker, image capturing arrangement, microphone, wearable device, and processing arrangement are operatively coupled to the cloud server, and wherein caregiver is authorised to access cloud server. Disclosed also is an in-vehicle cloud-based system for real-time monitoring of a subject in vehicle.

### TECHNICAL FIELD

The present disclosure relates generally to digital safeguarding; and more specifically, to a cloud-based system for real-time monitoring of a subject. The present disclosure also relates to an in-vehicle cloud-based system for real-time monitoring of a subject in the vehicle.

### BACKGROUND

Over the past decade, the technological advancement in smart sensing devices has extended an ability to care for others by providing digital safeguarding to the needy, worldwide, especially in healthcare sectors such as for the indoor patients as well as for patients-on-the move (referred to as a subject hereafter). Typically, the smart sensing device may be implemented as a closed-circuit television (CCTV) system, for monitoring the activity of the subject. However, surveillance through the CCTV system is not configured to provide preventive monitoring of the subject in the real-time and also renders the subject and other people in vicinity to be in a privacy-free environment 24×7.

Recently, the smart sensing devices may be combined with health monitoring systems into a personalized wearable health monitoring device that allows a real-time monitoring of the patient or subject. Typically, the wearable health monitoring devices measure and monitor heartbeat per minute, blood pressure, body temperature, body mass index, and other physiological information in real-time. However, the available wearable health monitoring devices fail to recognize an emergency situation and elicit a preventive action therefor.

Moreover, the smart sensing devices combined with the health monitoring system may be mounted within a vehicle, transporting the subject from one place to another, to monitor the health parameters thereof while on the move. Typically, the conventional in-vehicle health monitoring system also fail to predict the health condition and the preventive medical attention required to circumvent an emergency situation.

Therefore, in light of the foregoing discussion, there exists a need to overcome the aforementioned drawbacks associated with conventional health monitoring system.

### Summary

The present disclosure seeks to provide a cloud-based system for real-time monitoring of a subject. The present disclosure also seeks to provide an in-vehicle cloud-based system for real-time monitoring of a subject in the vehicle. The present disclosure seeks to provide a solution to the existing problem of health monitoring system. An aim of the present disclosure is to provide a solution that overcomes at least partially the problems encountered in prior art, and provides an efficient, reliable, and portable system for preventive monitoring of the subject.

In one aspect, an embodiment of the present disclosure provides a cloud-based system for real-time monitoring of a subject, the system comprising:
a real-time location tracker to track a location of the subject;
an image capturing arrangement configured to:
   capture an image data of the subject, and
   provide the image data to a cloud server;
a microphone configured to provide a two-way audio communication between the subject and a caregiver;
a wearable device, when in use by the subject, configured to provide a real-time medical data of the subject to the caregiver; and
a processing arrangement operatively coupled to the wearable device, the processing arrangement configured to, based on the real-time medical data,
   predict a health condition of the subject,
   monitor a progression of the health condition, and
   generate an alert based on the progression of the health condition, and
   advice, based on the generated alert, an action to be performed,
wherein the at least one of: the real-time location tracker, the image capturing arrangement, the microphone, the wearable device, and the processing arrangement are operatively coupled to the cloud server, and wherein the caregiver is authorised to access the cloud server.

In another aspect, an embodiment of the present disclosure provides an in-vehicle cloud-based system for real-time monitoring of a subject in the vehicle, the in-vehicle cloud-based system comprising:
a real-time location tracker to track a location of the subject;
an image capturing arrangement configured to:
   capture an image data of the subject, and
   provide the image data to a cloud server;
a microphone configured to provide a two-way audio communication between the subject and a caregiver;
a wearable device, when in use by the subject, configured to provide a real-time medical data of the subject to the caregiver; and
a processing arrangement operatively coupled to the wearable device, the processing arrangement configured to, based on the real-time medical data,
   predict a health condition of the subject,
   monitor a progression of the health condition, and
   generate an alert based on the progression of the health condition, and
   advice, based on the generated alert, an action to be performed,
wherein the at least one of: the real-time location tracker, the image capturing arrangement, the microphone, the wearable device, and the processing arrangement are operatively coupled to the cloud server, and wherein the caregiver is authorised to access the cloud server.

In yet another aspect, an embodiment of the present disclosure provides a software application, associated with a user device, to be executed by a processing arrangement of the user device, wherein the software application comprises:
receiving an input by a user, on a graphical user interface of the user device, for requesting a vehicle to ride a subject to a destination;
receiving a confirmation associated with completion of the ride to the destination by the processing arrangement; and
receiving an input by the user, on a graphical user interface of the user device, for providing feedback on the ride.

Embodiments of the present disclosure substantially eliminate or at least partially address the aforementioned problems in the prior art, and enable prediction of the health condition of the subject in real-time, by employing machine learning and artificial intelligence, and advising preventive measures to effectively manage for example a deteriorating health condition. Moreover, the disclosed system enables the disabled and elderly patients with complex health condition to travel with ease and under a robust digital safeguard.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a cloud-based system for real-time monitoring of a subject, in accordance with an embodiment of the present disclosure;

FIG. 2 is a block diagram of an in-vehicle cloud-based system for real-time monitoring of the subject in the vehicle, in accordance with an embodiment of the present disclosure;

FIG. 3 is a schematic illustration of a graphical user interface of the user device for real-time monitoring of a subject, in accordance with an embodiment of the present disclosure;

FIG. 4 is an exemplary illustration of requesting a vehicle for transporting the subject, in accordance with an embodiment of the present disclosure; and

FIG. 5 is an exemplary illustration of a vehicle booking journey, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a cloud-based system for real-time monitoring of a subject, the system comprising:
a real-time location tracker to track a location of the subject;
an image capturing arrangement configured to:
   capture an image data of the subject, and
   provide the image data to a cloud server;
a microphone configured to provide a two-way audio communication between the subject and a caregiver;
a wearable device, when in use by the subject, configured to provide a real-time medical data of the subject to the caregiver; and
a processing arrangement operatively coupled to the wearable device, the processing arrangement configured to, based on the real-time medical data,
   predict a health condition of the subject,
   monitor a progression of the health condition, and
   generate an alert based on the progression of the health condition, and
   advice, based on the generated alert, an action to be performed,
wherein the at least one of: the real-time location tracker, the image capturing arrangement, the microphone, the wearable device, and the processing arrangement are operatively coupled to the cloud server, and wherein the caregiver is authorised to access the cloud server.

In another aspect, an embodiment of the present disclosure provides an in-vehicle cloud-based system for real-time monitoring of a subject in the vehicle, the in-vehicle cloud-based system comprising:
a real-time location tracker to track a location of the subject;
an image capturing arrangement configured to:
   capture an image data of the subject, and
   provide the image data to a cloud server;
a microphone configured to provide a two-way audio communication between the subject and a caregiver;
a wearable device, when in use by the subject, configured to provide a real-time medical data of the subject to the caregiver; and
a processing arrangement operatively coupled to the wearable device, the processing arrangement configured to, based on the real-time medical data,
   predict a health condition of the subject,
   monitor a progression of the health condition, and
   generate an alert based on the progression of the health condition, and
   advice, based on the generated alert, an action to be performed,
wherein the at least one of: the real-time location tracker, the image capturing arrangement, the microphone, the wearable device, and theprocessing arrangement are operatively coupled to the cloud server, and wherein the caregiver is authorised to access the cloud server.

In yet another aspect, an embodiment of the present disclosure provides a software application, associated with a user device, to be executed by a processing arrangement of the user device, wherein the software application comprises:
receiving an input by a user, on a graphical user interface of the user device, for requesting a vehicle to ride a subject to a destination;
receiving a confirmation associated with completion of the ride to the destination by the processing arrangement; and
receiving an input by the user, on a graphical user interface of the user device, for providing feedback on the ride.

The present disclosure provides the aforementioned cloud-based system for real-time monitoring of a subject and the aforementioned in-vehicle cloud-based system for real-time monitoring of a subject in the vehicle. The in-vehicle cloud-based system for real-time monitoring of a subject during transportation of the subject from one place to another. In this regard, the cloud-based system enables predicting the health condition of the subject. Beneficially, the cloud-based system access the information stored in the cloud to predict a deterioration of health condition and provide remote monitoring and deploying the health facility when needed. In this regard, the cloud-based system employ a real-time tracking, a live video streaming and a real-time communication means for preventive monitoring of the subject. Moreover, the in-vehicle cloud-based system improves journey safety for the subject and helps save up to 60% of the transportation cost. Furthermore, the system has been designed compactly to support arrangement in care homes as well as on-the-move in vehicles.

The disclosed system provides a solution for monitoring the subject in real-time when the caregiver is at a remote location. Throughout the present disclosure, the term “monitoring” as used herein refers to a regular observation and recording of various activities performed by the subject to ensure quality of life and determine the real-time medical data thereof as a result of said activities. Optionally, the subject could be monitored when in, a home, a care home, a hospital, a workplace, an isolation, travelling (on-the-move), and the like. It will be appreciated that the aforementioned system enables the caregiver or the subject to monitor the medical data of the subject in line with information such as a diagnosis, a treatment, prescription drugs, laboratory tests, physiologic monitoring data, a need for hospitalization, and patient insurance from a past medical history of the subject.

The term “subject” as used herein refers to a person, such as a patient, an elderly person, a worker, a child, a sportsperson, and the like. The term “caregiver” as used herein refers to a person such as a family member, a guardian, a doctor, a medical practitioner. For example, the subject is a patient in a hospital and a nurse is the caregiver taking care thereof. Optionally, the subject is an elderly person in a care home or their home, and a care home manager or a family member is a caregiver to such elderly person.

The term “real-time monitoring” as used herein refers to tracking and recording continuously updated information about the health (namely, heath data) of the subject that may be remotely located in a hospital, a home, a care home, a medical centre and the like. Such medical data may be accessed and used by the caregiver to provide medical diagnosis and the corresponding treatments to the subject in order to prevent an emergency situation. Notably, the medical data of the subject can be accessed wirelessly via Internet (such as Wi-Fi, LAN, and the like). Moreover, the cloud-based system (Amazon Web Services, AWS) is used for storing, managing, processing and making available for accessing the medical information therein under superior data security.

The term “cloud-based system” as used herein refers to a structure and/or module that includes programmable and/or non-programmable components configured to store, process and/or share hosted services via the internet. Specifically, the cloud-based system includes any arrangement of physical or virtual computational entities capable of enhancing information to perform various computational tasks. Furthermore, it should be appreciated that the cloud-based system may be both a single hardware server and/or a plurality of hardware servers operating in a parallel or distributed architecture. In an example, the cloud-based system may include components such as a memory, a processor, a network adapter and the like, to store, process and/or share information with the caregiver. The cloud-based system offers a flexible solution that allows healthcare professionals and hospitals to leverage a network of remotely accessible servers for storing and accessing therefrom large volumes of data (namely, the medical data) in a secure environment. The disclosed cloud-based system is a private cloud-based system (devoted to a single business) or a hybrid cloud-based system that enables accessing the stored medical data associated with the subject with a small group of individuals with limited access and rights.

The term “real-time location tracker” as used herein refers to a tracking system that is used to track the position of the subject in real-time. The real-time location tracker is configured to provide the real-time positional information of the subject and upload it to the cloud server. Typically, the real-time positional information may include but is not limited to a coordinate, a postal code, an address, a region, an area, a locality.

The term “image capturing arrangement” as used herein refers to an arrangement that captures the movement of the subject. Optionally, the image capturing arrangement may have one or more image sensors that may be used to capture the image data. The term “image data” as used herein refers to visual representations of the subject, captured by the imaging capturing arrangement. Typically, the image data reflects a graphic representation and is used to store remotely sensed imagery, e.g. activity of the subject, a pose of the subject, and the like. Optionally, the image data comprises one or more images, one or more skeletal poses, or a video data. Optionally, the video data may be a collection of one or more images, time-stamped skeletal poses or succession of skeletal poses of the subject. Optionally the image capturing arrangement may be configured to detect the skeletal poses of the subject as the image data. Typically, one or more skeletal poses is a set of coordinates that can be connected to describe the pose of the subject.

Optionally, the image capturing arrangement may include but is not limited to, a camera, a night vision camera, a fish-eye camera, a wide-angle camera, a radar, an infrared camera, a lidar, an ultrasonic sensor and the like. Typically, the image capturing arrangement employs optical imaging systems (that use any of visible, near-infrared, and shortwave infrared spectrums and typically produce panchromatic, multispectral, and hyperspectral imagery), thermal imaging systems (that use mid to longwave infrared wavelengths), or synthetic aperture radar (SAR). Optionally, the image capturing arrangement can employ emission or transmission of energy in the form of waves (namely, waveform), such as light, particles, sound, and others. The waveform may interact with an object (or subject) in several ways, such as transmission, reflection, and absorption.

Optionally, the image capturing arrangement is configured to provide a live video data of the subject to the cloud server that is accessible by the caregiver. The image capturing arrangement is configured to analyze the activity of the subject by capturing the image data and providing the image data in real-time to the caregiver. In this regard, the image capturing arrangement captures the image data of the subject and provides the data to the cloud server which is accessible by the caregiver.

Optionally, the video data, comprising a stream of plurality of images together, is stored in the cloud server associated with the cloud-based system. It will be appreciated that the caregiver is authorized to access the video data to monitor the subject in real-time. In an example, the caregiver may be at a remote location, such as a hospital, and the subject could be at their home or a care home. It would have been difficult traditionally for the caregiver to know the health condition of the subject, however, the disclosed cloud-based system enables the caregiver to access the cloud server through a mobile application to monitor the subject in real-time based on the saved live video data of the subject on the cloud server.

The term “cloud server” as used herein refers to a powerful physical or virtual infrastructure that has been virtualized, to perform application-and information-processing storage and enable accessing of the stored information by users remotely over a network. The cloud server include suitable logic, circuitry, interfaces, and/or code that is configured to store, process and/or receive information from the real-time location tracker, the image capturing arrangement, the microphone, the wearable device and the processing arrangement. It will be appreciated that the cloud server may be both a single server and/or a plurality of servers operating in a parallel or distributed architecture to operatively couple with the disclosed cloud-based system or similar systems. Examples of the cloud server include, but is not limited to, a storage server, a web server, an application server, or a combination thereof.

The term “microphone” as used herein refers to a device (a transducer) that senses the sound and converts the sensed sound into an electrical signal. It will be appreciated that the microphone is configured to provide a two-way audio communication between the subject and the caregiver. Moreover, the microphone is used for transferring the audio of the subject to the caregiver and vice versa. The microphone may include but is not limited to a dynamic microphone, a condenser microphone, a diaphragm a microphone, and a ribbon microphone. Optionally the microphone is configured with a noise reduction module to remove the background noise during a communication. For example, in case the subject has dementia or severe memory problems (Alzheimer’s Society, 2019) and subject finds travelling challenging, disorientating, and frightening, the subject may communicate, by using the microphone, with the caregiver who is familiar with the subject and provides reassurance to the subject while travelling.

Optionally, system comprises a sim to enable the two-way audio communication over a mobile network between the subject and a caregiver. The term “sim” (short for the subscriber identity module) as used herein refers to a smart card that is configured to provide the user to connect to the mobile network. Typically, the sim stores identification information of a specific mobile network data that includes user identity, location and phone number, network authorization data, personal security keys, contact lists and stored text messages. The term “mobile network” as used herein refers to a communication network that links a group of devices to each other thereby enabling the devices to communicate with each other and share information. In an example, the mobile network includes but not limited to, a Global System for Mobile Communications (GSM), Code-Division Multiple Access (CDMA), Time-Division Multiple Access (TDMA), Frequency-Division Multiple Access (FDMA), Space-Division Multiple Access (SDMA), Advanced Mobile Phone System (AMPS), General Packet Radio Service (GPRS), Evolution-Data Optimized (EV-DO), Enhanced Data Rates for GSM Evolution (EDGE), Universal Mobile Telecommunications System (UMTS), Digital Enhanced Cordless Telecommunications (DECT), Integrated Digital Enhanced Network (iDEN), 2G, 3G, 4G, 5G, LTE, radio waves, and so on.

The term “wearable device” as used herein refers to a smart electronic device that is worn close to and/or on the surface of the skin, and detects, analyses, and transmits medical data of the subject, such as physiological information and/or ambient data, and allows, in some cases, immediate biofeedback to the caregiver and the subject. The term “real-time medical data” as used herein refers to health-related information that is associated with the subject in real-time and is continuously measured and monitored by the wearable device.

Optionally, the medical data is at least one of: a body temperature, a pulse rate, a blood pressure, a body mass index, a respiratory rate, an oxygen saturation, a pupil reaction, a state of consciousness. It will be appreciated that the medical data is measured and monitored by the wearable device, and is stored on the cloud server from where the authorised user, i.e. the caregiver, can access the stored medical data to monitor the subject and provide the necessary medical assistance if required. Optionally, the wearable device is configured to send alerts to the caregiver when any of the medical data reaches a critical stage. Optionally the wearable device is configured for tracking by an application software, when in operation. It will be appreciated that the application software is configured to send alerts to the caregiver. Optionally, the wearable device is communicably coupled to the user device via any one of: Bluetooth®, near field communication, Wi-Fi. Optionally, the medical data may be shared by the wearable device to the user device by using a media access control address (MAC address or MAC Id).

Optionally, the wearable device is configured to be attachable to, detachable from and/or reattachable to a body part of the subject. Optionally, the body part is the skin of: head, sternum, arm, shoulder, wrist, hand, neck, trunk, ankle, leg, thigh, foot, temple, face, neck, elbow, wrist, fingers, spine, knee, and so forth. Alternatively, optionally, the wearable device may be a handheld device or configured to be attachable to, detachable from and/or reattachable to a cloth or a footwear of the subject. Optionally, the physical contact with the subject is by a mechanical engagement means, selected from at least one of: an adhesive, a strap, a locket, a bracelet, a band, a belt, a vacuum cup, a magnet, and a hook and loop fastener. It will be appreciated that the wearable device is wearable over any body part of the subject or placed at a suitable location, such as on a shirt, footwear, headband, and so on, rather than a slip-on arrangement. For example, the wearable device may be placed upon the skin directly (for example using adhesives, vacuum cups, and so on) or incorporated into one of a bracelet, a pendant, an anklet, an armband, a wrist band, clothes, footwear, or other item such that the proximal surface thereof is arranged facing the skin of the subject.

Optionally, the wearable device comprises an electric charging portion to supply an onboard battery that powers the wearable device. Optionally, the onboard battery is a 2200 mAh 7.4 V Li-Po battery that provides power backup for about 6-7 hours. Optionally, the electric charging portion is configured to receive electric power (for example electric power) from an external electric power source by a wireless or wired connection.

The term “processing arrangement” as used herein refers to a set of algorithms, an application, a program, a process, or a device that responds to requests for information or services by another application, program, process or device (such as the external device) via a network interface. Optionally, the processing arrangement also encompasses software that makes the act of serving information or providing services possible. It may be evident that the communication means of an external device may be compatible with a communication means of the processing arrangement, in order to facilitate communication therebetween. Optionally, the processing arrangement employs information processing paradigms such as artificial intelligence, machine learning, cognitive modelling, and neural networks for performing various tasks associated with real-time health monitoring of the subject. Moreover, the processing arrangement is operatively coupled to the wearable device. Furthermore, the processing arrangement is configured to predict a health condition of the subject based on the real-time medical data and monitor a progression of the health condition, generate an alert based on the progression of the health condition, and advice, based on the generated alert, an action to be performed.

The term “predict” as used herein refers to anticipation of the health of the subject based on the medical data, that may be indicated to the user (caregiver of subject) via a software application on the graphical user interface of the user device. The term “progression” as used herein refers to a state of deterioration or improvement of the health of the subject over a period of time in which real-time monitoring of the subject is being performed. The term “alert” as used herein refers to a notification corresponding to a deterioration of the health of the subject, which is sent to the caregiver on the user device to perform a desired action to circumvent the emergency situation. It will be appreciated that the alert algorithm will prevent any future and further deterioration of the subject’s medical condition while in transport or in care homes. Optionally, the alerts may be generated to the user (caregiver of subject) via the software application on the graphical user interface of the user device. Optionally, the alert may be a short message service (SMS) directed at the user device. In this regard, the SMS notification may provide instant alerts about the tracked object (vehicle and/or the subject). Typically, the actions to be performed may include, but do not limit to, speaking with the patient, providing first aid, attaching the subject to a support-machine, giving cardiopulmonary resuscitation (CPR), providing insulin (in case blood glucose level shoots), and so forth.

Optionally, the cloud server may be communicably coupled with the user device via a wired and/or a wireless data communication network. In an example, the communication network includes but not limited to, a cellular network, short range radio (for example, such as Bluetooth®), Internet, a wireless local area network, and an Infrared Local Area Network, or any combination thereof. Optionally, the cloud server may be within the user device. Optionally, the user device is a mobile phone or a smart phone. Optionally, the cloud server may store history and reports associated with the GPS data (such as notifications, and so on).

Optionally, the communication network may be an inbuilt Wi-Fi, an inbuilt Bluetooth (Bluetooth low energy (BLE) technology/on-board diagnostic (OBD) tool), a 4G or 5G module, an embedded sim (eSIM), an over-the-air (OTA) firmware, and so forth. Notably, in case of the inbuilt Wi-Fi, the processing arrangement may provide (or act as) a Wi-Fi hotspot that passengers can use as an access for the internet surfing. Moreover, such Wi-Fi hotspots may have other ports for connecting wireless devices like the IP imaging device (namely IP camera). The inbuilt Bluetooth server enables connecting BLE devices to OBD. The 5G/4G modem may be used to provide internet access to send the medical data and so on to cloud server as well as for connecting to the Wi-Fi hotspot. The eSIM provides a provision enabled for internet instead of 5G/4G module. The OTA firmware can be updated to enable and disable one or more features of the cloud-based system according to various versions by the binary file.

Optionally, the processing arrangement is trained using machine learning and artificial intelligence, to
predict the health condition of the subject,
monitor the progression of the health condition,
generate an alert based on the progression of the health condition, and
advice, based on the generated alert, an action to be performed.

In this regard, the machine learning and artificial intelligence is used to train the processing arrangement to monitor and predict the health condition of the subject to determine a potential deterioration (or improvement) in the health over time. The term “machine learning algorithm” as used herein refers to a subset of artificial intelligence (AI) in which algorithms are trained using training datasets. For example, the training dataset may be a historical data, such as image data, medical data, and so forth, stored in a memory module of the cloud-based system or the cloud server, to predict outcomes, future trends and draw inferences from patterns of the historical data. The term “artificial intelligence tools” as used herein relates to a computationally intelligent system that combines knowledge, techniques, and methodologies for controlling a bot or other programmable arrangements within a computing environment. Furthermore, the artificial intelligence system is configured to apply the knowledge that can adapt itself and learn to do better in changing environments. The artificial intelligence tools include fuzzy logic engines, decision-making engines, pre-set targeting accuracy levels, and/or programmatically intelligent software. Optionally, the artificial intelligence tools enable data gathering of various people globally to provide consistent mapping of the potential health condition with a current (namely, real-time) health condition of the subject. In other words, disclosed system employs predictive analysis to prevent further medical and physical deterioration while transporting the subject or while the subject is in the care home.

In one example, if for a subject, the health condition changes substantially from a routine, the machine learning algorithms may raise an alert (a flag or an alarm) to the caregiver. In this regard, the processing arrangement continually collects several data points from the wearable device, the microphone, the image capturing arrangement and the real-time location tracker monitoring the subject in real-time, for predicting the health condition, monitoring the progression of the health condition, generating an alert based on the progression of the health condition, and advising, based on the generated alert, the action to be performed, and the machine learning algorithm or other artificial intelligence tools to train the processing arrangement for future such scenarios.

Typically, such algorithms are a step-by-step computational procedure for solving a problem, similar to decision-making flowcharts, which are used for information processing, mathematical calculation, and other related operations. Notably, the aforementioned algorithms reduce the computational complexity and provide powerful computing units to process the medical data and monitor the subject(s) in real-time. Moreover, the aforementioned algorithms also help the cloud-based system to estimate a potential medical condition from any historical data. Beneficially, the aforementioned algorithms improve the performance of the cloud-based system by reducing the time required for a preventive monitoring of the subject by conventional methods.

It will be appreciated that the cloud-based system may comprise an extendable memory therein. Optionally, such extendable memory may be an SD card for example. Optionally, the SD card may store up to 256GB data within the cloud-based system when the internet connection is not available. Moreover, the stored data may be transferred (pushed) to the cloud server once the internet connection is established between the cloud-based system and the cloud server.

The present disclosure also relates to the in-vehicle cloud-based system as described above. Various embodiments and variants disclosed above apply mutatis mutandis to the in-vehicle cloud-based system.

The term “in-vehicle cloud-based system” as used herein refers to the aforementioned cloud-based system that is configured within a vehicle. The vehicle can be an ambulance, a cab, and the like having medical equipment to support the subject while on-the-move (i.e. transportation) from one place to another. The medical equipment is configured to provide medical assistance to the subject during transportation of the subject. Moreover, the vehicle is installed with the aforementioned cloud-based system (now referred to as the ‘in-vehicle cloud-based system’) configured to provide the in-vehicle data to the caregiver. Moreover, the in-vehicle cloud-based system provides ease and confidence to the disabled and elderly subjects with complex health condition who need to travel to the hospital for regular check-ups appointment or in case of emergency to transport from one location to the care facility, along with providing the real-time monitoring of the subject’s medical data for access by the caregiver.

Optionally, the real-time location tracker utilizes a navigation system and an onboard vehicle diagnostic to monitor journey data. It will be appreciated that the real-time location tracker in the vehicle tracks the real-time location of the vehicle transporting the subject. The navigation system is a real-time map of the current location and step-by-step directions to a requested location. Optionally, the navigation systems may be on the vehicle or within the in-vehicle cloud-based system. Notably, the navigation system provides the shortest path between the location to transport the patient quickly.

Optionally, the navigation system is a global positioning system. In this regard, the global positioning system (GPS) is the satellite-based radio navigation system configured to perform calculations about vehicle speed, the travel time between locations, and the estimated time of arrival of the vehicle. Optionally, the navigation system can also use BeiDou Navigation Satellite System (BDS), Global Navigation Satellite System (GLONASS), Galileo, Indian Regional Navigation Satellite System (IRNSS)/Navigation Indian Constellation (NavIC), Quasi-Zenith Satellite System (QZSS).

Optionally, onboard vehicle diagnostic comprises vehicle data selected from at least one of: a registration number, a name of a driver, a speed, a list of medical equipment, a license number, and at least one service rating. The onboard vehicle diagnostic is used by a user to verify the vehicle data of the vehicle. Optionally the application is used to check the onboard vehicle diagnostic. In this regard, the registration number is the number attached to the vehicle for official identification purposes. Typically, the registration number is printed on a number plate, a license plate (a metal or plastic plate attached to the vehicle on the front and rear side thereof). Moreover, the driver’s name and the driving license is provided to be verified by the caregiver. Furthermore, the list of medical equipment comprises the equipment including, but not limited to, a stretcher, a wheelchair, an oxygen cylinder, an oximeter, a temperature measuring device, a blood pressure monitoring device, an electrocardiogram machine.

Optionally, the vehicle comprises at least one occupant to assist the subject in the vehicle during transportation. The at least one occupant may be any one of: the caregiver, a paramedical staff, a healthcare professional (such as a nurse), a driver of the vehicle, an assistant to the driver. The transportation of the subject for example can be from a home or care home to the hospital and vice versa.

Optionally, the image capturing arrangement comprises a first imaging device to capture an environment ahead of the vehicle; and a second imaging device to capture at least a portion inside the vehicle. In this regard, the first imaging device is placed in a front cabin of the vehicle to capture the image data of the environment in front of the vehicle to access the traffic, bumps on the road, for example. Moreover, the front cabin is configured to include a seat for the driver, a passenger seat adjacent to the driver’s seat, an accelerometer, a speedometer, a speed sensing device located on the vehicle, one or more medical equipment, and a first imaging device. The second imaging device is placed in a rear cabin of the vehicle to capture the image data of the patient, the caregiver and a rear environment of the vehicle. Moreover, the rear cabin is configured to accept the subject, the caregiver, and a set-up of one or more medical equipment. In an embodiment, the second imaging device may be arranged in the vehicle to capture both the environments of the front cabin and the second cabin. In such case, the second imaging device may be a fish-eye camera providing a180°-180° coverage.

It will be appreciated that the imaging arrangement may comprise more than one imaging device, such as the first imaging device, the second device, a third imaging device and a fourth imaging device.

Optionally, the image capturing arrangement is an internet protocol (IP) camera. In this regard, at least one of the first imaging device and the second imaging device is an IP camera. Notably, the IP camera is a digital security camera that receives and sends images (and/or video) via an IP network.

The present disclosure also relates to the software application as described above. Various embodiments and variants disclosed above apply mutatis mutandis to the software application.

Typically, the software application is a computer program or a group of programs designed to run (locally or through a web browser) on the user device associated with the user, such as the subject, or any caregiver using the user device. Optionally, the application software may be affiliated to an organization, for example, the hospital or health provider service. Therefore, the application software functions in accordance with pre-programmed guidelines provided by the organization. The software application is designed to suit compatible device, such as android or iOS. Moreover, the software application is designed to help users to perform an activity, such as health management, health monitoring, and so on, for which the software application can manipulate at least one of: a text, numbers, an audio, graphics or any combination thereof. In this regard, the user device provides a user interface on the display thereof for executing the software application. The software application is accessible by the graphical user interface of the user device.

The software application is communicably, operatively coupled to the cloud server associated with the cloud-bases system. Typically, via the software application, the user sends the request for the vehicle. Optionally, the graphical user interface displays a list of available vehicles. The user may select a desired vehicle from the list of available vehicles after ensuring that the vehicle comprises one or more desired medical equipment therein to support the health condition of the subject during transportation thereof.

Moreover, the software application receives a confirmation by the processing arrangement after the completion of the ride as soon as the vehicle reaches the destination. Furthermore, the software application receives the input by the user, on a graphical user interface of the user device, for providing feedback on the ride. In this regard, the processing arrangement is communicably coupled with the user device, associated with the user. Specifically, the processing arrangement may be communicably coupled with the user device using a wired and/or a wireless data communication network. Optionally, there is a separate processing arrangement associated with the user device, wherein the processing arrangement of the user device is operable to communicate with the processing arrangement of the cloud-based system. Optionally, the user device is a mobile phone, a smart phone, a computer, a laptop. The software application is configured to function in accordance with the pre-programmed guidelines upon installation thereof.

Optionally, the software application is configured to relay an alert based on a progression of a health condition of a subject. It will be appreciated that the alert algorithm will prevent any future and further deterioration of the subject’s medical condition while in transport or in care homes.

Optionally, the alert is relayed to at least one of: a vehicle service unit, the user, and a healthcare provider associated with the destination. It will be appreciated that the healthcare provider may be a hospital, a doctor or a care home manager.

### DETAILED DESCRIPTION OF THE DRAWINGS

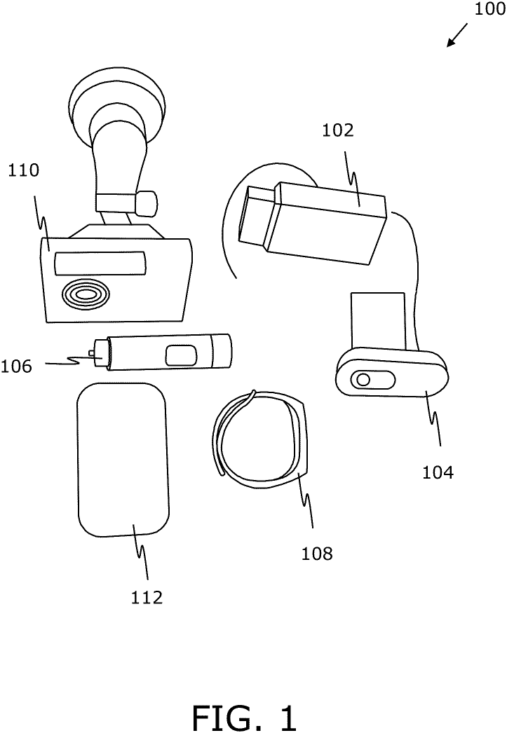

Referring to FIG. 1, illustrated is a cloud-based system 100 for real-time monitoring of a subject in accordance with an embodiment of the present disclosure. The system 100 comprises a real-time location tracker 102 to track a location of the subject. The system 100 also comprises an image capturing arrangement 104 configured to capture an image data of the subject, and to provide the image data to a cloud server (not shown). Moreover, the system 100 also comprises a microphone 106 configured to provide a two-way audio communication between the subject and a caregiver and a wearable device 108, when in use by the subject, configured to provide a real-time medical data of the subject to the caregiver. The system 100 also comprises a processing arrangement 110 operatively coupled to the wearable device 108, the processing arrangement 110 is configured to, based on the real-time medical data, to predict a health condition of the subject, to monitor a progression of the health condition, generate an alert based on the progression of the health condition, and advice, based on the generated alert, an action to be performed.

Moreover, the processing arrangement 110 is operatively coupled to a communication means 112 to facilitate communication therebetween. The communication means 112 provide network to the real-time location tracker 102, the image capturing arrangement 104, the microphone 106, the wearable device 108, and the processing arrangement 110 to operatively coupled to the cloud server. Furthermore, the processing arrangement 110 is operatively coupled to the cloud server, and wherein the caregiver is authorised to access the cloud server.

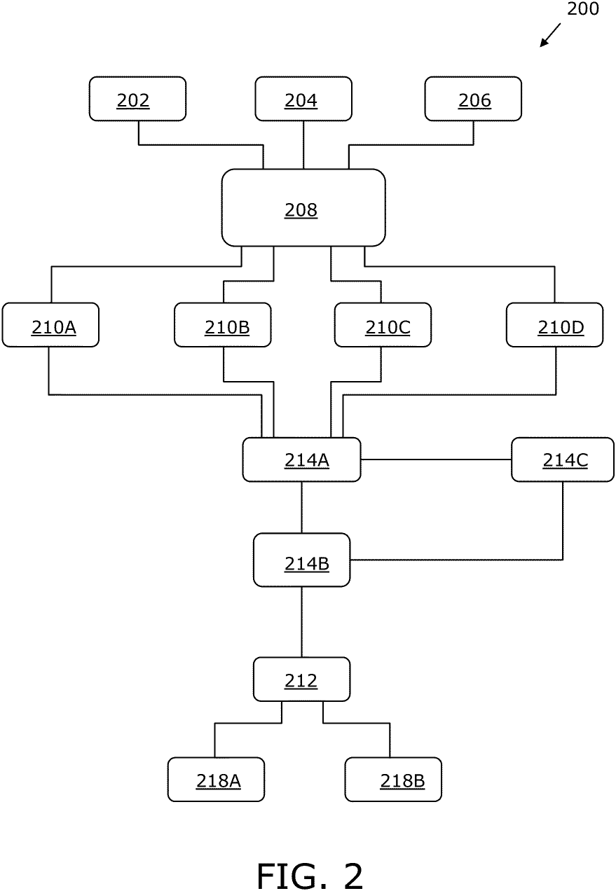

Referring to FIG. 2, illustrated is a block diagram of an in-vehicle cloud-based system 200 for real-time monitoring of the subject in the vehicle, in accordance with an embodiment of the present disclosure. As shown, the in-vehicle cloud-based system 200 comprises a real-time location tracker 202 to track a location of the subject. The real-time location tracker 202 may be implemented as a GPS. The in-vehicle cloud-based system 200 further comprises a Bluetooth 204, and a Wi-Fi 206. The Wi-Fi 206 is configured to connect one or more Wi-Fi-enabled devices, such as an imaging device (for example an IP camera) to a Wi-Fi hotspot and/or enable connecting a user device to a Wi-Fi hotspot for internet surfing. Moreover, the in-vehicle cloud-based system 200 comprises a processing arrangement 208 and one or more imaging arrangement, such as the image capturing arrangements 210A, 210B, 210C and 210D. The image capturing arrangement is configured to: capture an image data of the subject, and provide the image data to a cloud server 212 via a communication network, implemented as internet connection 214A, a 4G/5G module 214B and/or a SD Card 214C. The SD Card 214C is configured to store a data of up to 256GB when the internet connectivity is not established (or lost) between the image capturing arrangement and the cloud server 212. Furthermore, the in-vehicle cloud-based system 200 comprises a microphone (not shown) configured to provide a two-way audio communication between the subject and a caregiver and a wearable device (not shown), when in use by the subject, configured to provide a real-time medical data of the subject to the caregiver. The processing arrangement 208 is operatively coupled to the wearable device, the processing arrangement 208 is configured to, based on the real-time medical data from the wearable device, predict a health condition of the subject, monitor a progression of the health condition, generate an alert based on the progression of the health condition, and advice, based on the generated alert, an action to be performed, wherein the at least one of: the real-time location tracker 202, the image capturing arrangement, such as the image capturing arrangements 210A, 210B, 210C and 210D, the microphone, the wearable device, and the processing arrangement 208 are operatively coupled to the cloud server 212, and wherein the caregiver is authorised to access the cloud server 212. The cloud server 212 is configured to provide a cloud data requested by a user (such as the caregiver) using a URL 216A and/or SMS or email services 216B.

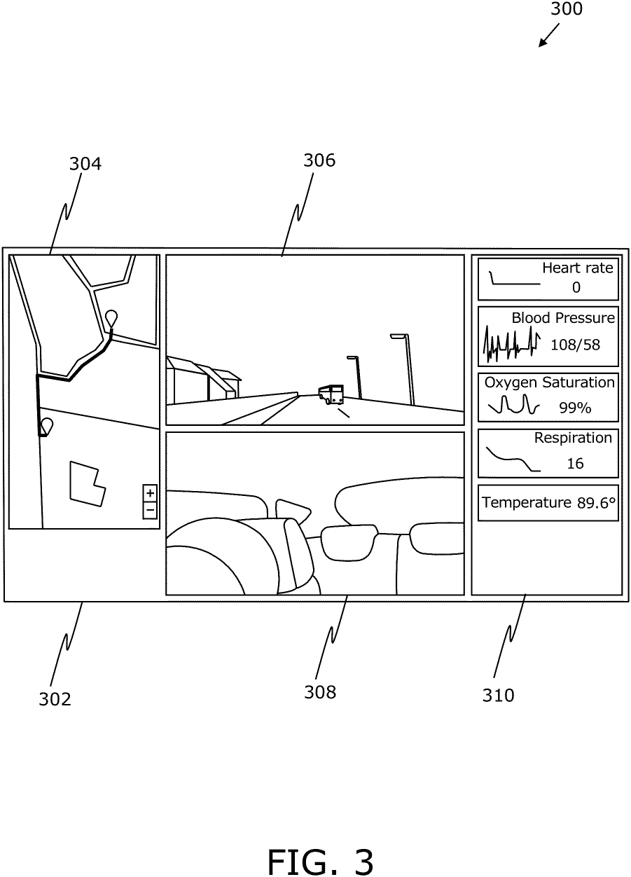

Referring to FIG. 3, illustrated is a schematic illustration of a graphical user interface 300 on a user device 302 for real-time monitoring of the subject, in accordance with an embodiment of the present disclosure. As shown the user device 302 is configured to display the real-time location tracking of the subject by utilizing a navigation system 304 to monitor journey data. The navigation system 304 provides the real-time location information of the subject to the caregiver to track the location thereof during transportation. Moreover, the user device 302 is configured to provide the image data captured by a first imaging device 306 and a second imaging device 308. The first imaging device 306 captures an environment ahead of the vehicle and the second imaging device 308 to capture at least a portion inside the vehicle. The user device 304 displays the real-time medical data 310. The real-time medical data 310 is configured to measure at least one of: a body temperature, a pulse rate, a blood pressure, a body mass index, a respiratory rate, an oxygen saturation, a pupil reaction, a state of consciousness of the subject to display on the user device 304.

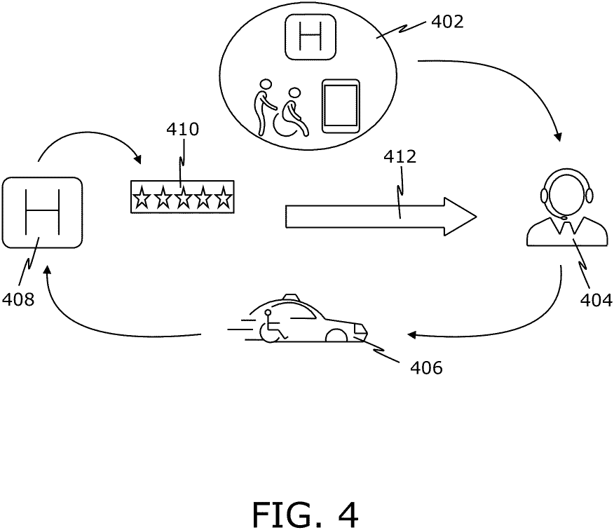

Referring to FIG. 4, illustrated is an exemplary illustration of requesting a vehicle for transporting the subject, in accordance with an embodiment of the present disclosure. At step 402, the user is configured to contact the vehicle on the graphical user interface from the user device. At step 404, an input from the user is received on a graphical user interface of the user device, for requesting the vehicle that will ride the subject to the destination. At step 406, the vehicle is received on the location and the caregiver provides the assistance to help the subject to get into the vehicle. At step 408, the vehicle having the subject therein is started to transport the subject to the destination, such as a hospital, a care home, a home, and the like. The user can monitor the travelling status on the graphical user interface of the user device. At step 410, the feedback of the journey is provided by the user. At step 412, the service request is registered by the graphical user interface of the user device to assist the user to book the vehicle in future in case of regular check-ups or emergency.

The steps 402, 404, 406, 408, 410 and 412 are only illustrative and other alternatives can also be provided where one or more steps are added, one or more steps are removed, or one or more steps are provided in a different sequence without departing from the scope of the claims herein.

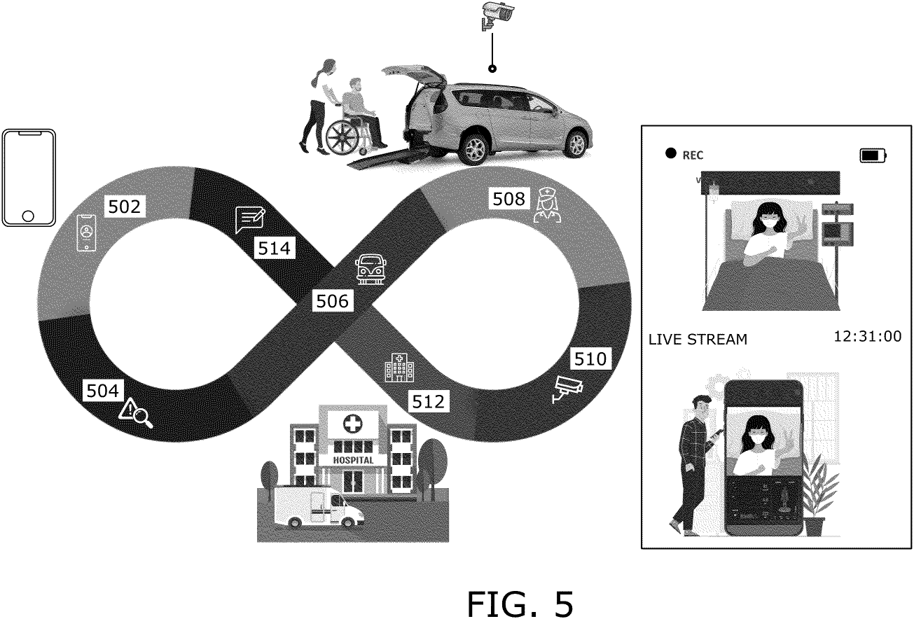

Referring to FIG. 5, illustrated is an exemplary illustration of a vehicle booking journey, in accordance with an embodiment of the present disclosure. At step 502, the user contacts the vehicle on the graphical user interface from the user device and provide the information related to the medical assistance needed by the subject. At step 504, provided information is analysed by the graphical user interface. At step 506, the vehicle is booked based on the provided information. At step 508, the vehicle arrives with trained caretaker, who assists the subject into and inside the vehicle. At step 510, the live streaming and monitoring of the subject during the journey is provided to the user. At step 512, the vehicle having the subject reached the destination, such as a hospital, a care home, a home, and the like. At step 514, the feedback of the journey is provided by the user to improve the journey.

The steps 502, 504, 506, 508, 510, 512 and 514 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A cloud-based system for real-time monitoring of a subject, the system comprising:
a real-time location tracker to track a location of the subject;
an image capturing arrangement configured to:
capture an image data of the subject, and
provide the image data to a cloud server;
a microphone configured to provide a two-way audio communication between the subject and a caregiver;
a wearable device, when in use by the subject, configured to provide a real-time medical data of the subject to the caregiver; and
a processing arrangement operatively coupled to the wearable device, the processing arrangement configured to, based on the real-time medical data,
predict a health condition of the subject,
monitor a progression of the health condition,
generate an alert based on the progression of the health condition, and
advice, based on the generated alert, an action to be performed,
wherein the at least one of: the real-time location tracker, the image capturing arrangement, the microphone, the wearable device, and the processing arrangement are operatively coupled to the cloud server, and wherein the caregiver is authorised to access the cloud server.

2. A system according to claim 1, wherein the processing arrangement is trained using machine learning and artificial intelligence, to
predict the health condition of the subject,
monitor the progression of the health condition,
generate an alert based on the progression of the health condition, and
advice, based on the generated alert, the action to be performed.

3. A system according to claim 1, wherein the medical data is at least one of: a body temperature, a pulse rate, a blood pressure, a body mass index, a respiratory rate, an oxygen saturation, a pupil reaction, a state of consciousness.

4. A system according to claim 1, wherein the image data comprises one or more images, one or more skeletal poses, or a video data.

5. A system according to claim 1, wherein the image capturing arrangement is configured to provide a live video data of the subject to the cloud server that is accessible by the caregiver.

6. A system according to claim 1, further comprises a sim to enable the two-way audio communication over a mobile network between the subject and a caregiver.

7. An in-vehicle cloud-based system for real-time monitoring of a subject in the vehicle, the in-vehicle cloud-based system comprising:
a real-time location tracker to track a location of the subject;
an image capturing arrangement configured to:
capture an image data of the subject, and
provide the image data to a cloud server;
a microphone configured to provide a two-way audio communication between the subject and a caregiver;
a wearable device, when in use by the subject, configured to provide a real-time medical data of the subject to the caregiver; and
a processing arrangement operatively coupled to the wearable device, the processing arrangement configured to, based on the real-time medical data,
predict a health condition of the subject,
monitor a progression of the health condition,
generate an alert based on the progression of the health condition, and
advice, based on the generated alert, an action to be performed,
wherein the at least one of: the real-time location tracker, the image capturing arrangement, the microphone, the wearable device, and the processing arrangement are operatively coupled to the cloud server, and wherein the caregiver is authorised to access the cloud server.

8. An in-vehicle cloud-based system according to claim 7, wherein the vehicle comprises at least one occupant to assist the subject in the vehicle during transportation.

9. An in-vehicle cloud-based system according to claim 7, wherein the real-time location tracker utilizes a navigation system and an onboard vehicle diagnostic to monitor journey data.

10. An in-vehicle cloud-based system according to claim 9, wherein the navigation system is a global positioning system.

11. An in-vehicle cloud-based system according to claim 9, wherein onboard vehicle diagnostic comprises vehicle data selected from at least one of: a registration number, a name of a driver, a speed, a list of medical equipment, a license number, and at least one service rating.

12. An in-vehicle cloud-based system according to claim 7, wherein the image capturing arrangement comprises:
a first imaging device to capture an environment ahead of the vehicle; and
a second imaging device to capture at least a portion inside the vehicle.

13. A software application, associated with a user device, to be executed by a processing arrangement of the user device, wherein the software application comprises:
receiving an input by a user, on a graphical user interface of the user device, for requesting a vehicle to ride a subject to a destination;
receiving a confirmation associated with completion of the ride to the destination by the processing arrangement; and
receiving an input by the user, on a graphical user interface of the user device, for providing feedback on the ride.

14. A software application according to claim 13, wherein the software application is configured to relay an alert based on a progression of a health condition of a subject.

15. A software application according to claim 13, wherein the alert is relayed to at least one of: a vehicle service unit, the user, and a healthcare provider associated with the destination.
